(19) United States
(12) Patent Application Publication
(10) Pub. No.: US 2023/0140872 A1
(43) Pub. Date: May 11, 2023

(54) SPACER AND METHOD OF MENUFACTURING SAME

(71) Applicant: Smilecad Co., Ltd., Busan (KR)

(72) Inventors: Seok Joon KIM, Busan (KR); Jae Ho JEON, Busan (KR); Byeong Soo KIM, Busan (KR)

(73) Assignee: Smilecad Co., Ltd., Busan (KR)

(21) Appl. No.: 17/983,357

(22) Filed: Nov. 8, 2022

(30) Foreign Application Priority Data
Nov. 10, 2021 (KR) 10-2021-0154187

(51) Int. Cl. A61C 7/10

(52) U.S. Cl. CPC A61C 7/10; A61C 2201/007

(57) ABSTRACT

Proposed are a spacer and a method of manufacturing the spacer. The spacer includes a body portion configured as a single structure and formed of a shape memory material, the body portion supporting teeth at both sides of a tooth loss space. Furthermore, the body portion includes a first grip portion surrounding a part around a tooth disposed at a first side with respect to the tooth loss space, a second grip portion surrounding a part around a tooth disposed at a second side with respect to the tooth loss space, and a bridge connecting the first grip portion and the second grip portion to each other. Furthermore, the first grip portion and the second grip portion have curved shapes open toward directions opposite to each other. Furthermore, the method includes a data obtaining process, a data processing process, and a laser cutting process.

### CROSS REFERENCE TO RELATED APPLICATION

The present application claims priority to Korean Patent Application No. 10-2021-0154187, filed Nov. 10, 2021, the entire contents of which are incorporated herein for all purposes by this reference.

### BACKGROUND OF THE INVENTION

### Field of the Invention

The present disclosure relates to a spacer and a method of manufacturing the spacer. More specifically, the present disclosure relates to a spacer formed of a shape memory material, and to a method of manufacturing the spacer.

### Description of the Related Art

A spacer is an apparatus for maintaining a space so as to prevent a space loss at a position where a tooth is lost from occurring, and is referred to as a space-maintaining apparatus.

When the space loss occurs at the position where the tooth is lost, failure in an eruption of a permanent tooth may occur or difficulty in a treatment process such as an implant treatment may occur. Therefore, in order for the eruption of the permanent tooth, the treatment process, and so on, there is a necessity to prevent the space loss, and the spacer is applied to a dental treatment to satisfy the necessity.

A conventional spacer includes a band having a closed-loop shape so that the band is put in a tooth disposed at a first side of the tooth loss space and a loop that protrudes from the band so as to support a tooth disposed at a second side of the tooth loss space, and the conventional spacer is manufactured of a stainless steel material.

The loop is welded to the band, or is coupled to the band by a coupling member such as a screw. In addition, the loop is formed such that the loop surrounds around the tooth loss space so as to secure a space where the implant treatment is performed or the tooth is erupted. That is, the loop includes a pair of extension portions which crosses a lingual side and a buccal side of the tooth loss space and which has a first end coupled to the band, and includes a supporting portion which connects a second end that extends and which is in contact with and supports the tooth disposed at the second side of the tooth loss space.

In order to manufacture the conventional spacer as described above, a process in which a plaster solid of a patient's teeth arrangement is manufactured, then a loop and a band suitable for the patient's teeth is prepared and coupled, and then the band is put in the plaster solid and the loop is bent so as to manufacture the spacer suitable of the patient's tooth loss space is required to be performed. Accordingly, there are problems that a long time is required to manufacture the spacer, the manufacturing cost is increased, and the reduction in the tooth loss space is progressed during the manufacturing period occurs.

In addition, in the conventional spacer, as the band is formed such that the band surrounds the entire circumference of the tooth, an additional treatment process for securing a space where the band is inserted is required to be performed before the band is mounted on the tooth. Specifically, a treatment process for securing a space where the band is inserted is required to be performed by putting a rubber elastic body between the tooth where the band is mounted and an adjacent tooth, so that there is a problem that pain is caused since a pressure is applied to the teeth. That is, due to the structure of the band, there are problems that the treatment process becomes complicated and pain is caused to the patient.

Recently, a manufacturing process of a spacer has been reduced, and a spacer capable of being easily mounted has been developed.

In U.S. Pat. No. 9,339,351 B2 that is the related art, a spacer is proposed, the spacer being formed of a stainless steel material, the spacer having a loop (spacer 24) and a band (ring 12), and the spacer being capable of detachably coupling the loop to the band and being capable of adjusting a circumferential length of the band.

However, in the spacer in the document of the related art, since the band and the loop are also required to be separately manufactured and a configuration of the spacer is more complexed due to a structure for coupling the band to the loop, there is a problem that the spacer is still not conveniently manufactured.

In addition, in the spacer in the document of the related art, as the band is formed such that the band surrounds the entire circumference of the tooth, an additional treatment process for securing a space where the band is inserted is still required to be performed before the band is mounted on the tooth, and there are problems that pain is caused to a patient during performing the treatment process and the treatment period is increased.

### DOCUMENT OF RELATED ART

(Patent Document 1) U.S. Pat. No. 9,339,351 B2

### SUMMARY OF THE INVENTION

Accordingly, the present disclosure has been made keeping in mind the above problems occurring in the related art, and an objective of the present disclosure is to provide a spacer and a method of manufacturing the spacer capable of being easily manufactured as an integrated structure on the basis of scanning data of a teeth arrangement.

In addition, another objective of the present disclosure is to provide a spacer and a method of manufacturing the spacer in which an additional treatment process for securing a space to mount the spacer is not required to be performed.

In addition, still another objective of the present disclosure is to provide a spacer and a method of manufacturing the spacer in which interference of the spacer while a treatment is performed on a tooth loss space is minimized, the spacer being capable of effectively avoiding an occlusal force.

In order to achieve the above objectives, according to an embodiment of the present disclosure, there is provided a spacer including: a body portion configured as a single structure and formed of a shape memory material, the body portion supporting teeth at both sides of a tooth loss space, wherein the body portion includes: a first grip portion surrounding a part around a tooth disposed at a first side with respect to the tooth loss space; a second grip portion surrounding a part around a tooth disposed at a second side with respect to the tooth loss space; and a bridge connecting the first grip portion and the second grip portion to each other, and the first grip portion and the second grip portion have curved shapes open toward directions opposite to each other.

In addition, the bridge may be disposed to be lateralized from center portions of the first grip portion and the second grip portion such that the bridge crosses an outer side of the tooth loss space.

In addition, at least one of the first grip portion and the second grip portion may have both ends that extend so as to surround a corresponding tooth and a tooth adjacent to the corresponding tooth together.

In addition, the body portion may be manufactured by laser cutting a base material formed of a shape memory alloy material on the basis of 3D scanning data of a teeth arrangement, or may be manufactured by 3D printing the shape memory material on the basis of the 3D scanning data of the teeth arrangement.

In order to achieve the above objectives, according to an embodiment of the present disclosure, there is provided a method of manufacturing a spacer, the spacer including a plurality of grip portions formed such that the plurality of grip portions surrounds parts around teeth disposed at both sides of a tooth loss space so as to maintain the tooth loss space, the spacer including a bridge connecting the plurality of grip portions to each other, and the method including: a data obtaining process obtaining 3D scanning data of a teeth arrangement including the tooth loss space by using a 3D scanning device; a data processing process generating a plurality of grip portions processing lines surrounding the parts around the teeth disposed at the both sides of the tooth loss space and generating a bridge processing line connecting the plurality of grip portions processing lines to each other, on the 3D scanning data; and a laser cutting process laser cutting a base material having a flat plate shape formed of a shape memory alloy material on the basis of processing line data including the plurality of grip portions processing lines and the bridge processing line, thereby forming a body portion forming body having a basic shape of the body portion on the base material.

In the spacer and the method of manufacturing the spacer according to an embodiment of the present disclosure, following effects may be realized.

First, the spacer according to an embodiment of the present disclosure includes the body portion supporting the teeth at the both sides of the tooth loss space in order to maintain the tooth loss space. In addition, the body portion includes the plurality of grip portions surrounding parts around the teeth at the both sides of the tooth loss space, and includes the bridge that connects the plurality of grip portions to each other.

At this time, the plurality of grip portions has curved shapes open toward the directions opposite to each other. That is, the plurality of grip portions has shapes open toward the directions opposite to the tooth loss space. Therefore, unlike the conventional spacer that surrounds the entire circumference of the tooth, the additional treatment process for securing the space to mount the spacer between the adjacent teeth is not required to be performed, so that there is an advantage that inconvenience of the patient may be prevented from occurring and the treatment period may be reduced.

Second, the bridge is formed such that the bridge crosses the outer side of the tooth loss space so as to avoid the tooth loss space. That is, the bridge is disposed to be lateralized from the center portion of the grip portions.

Accordingly, interference does not occur on a tooth erupting from the tooth loss space and an implanted tooth, and there is an effect that the treatment may be more smoothly performed by effectively opening the tooth loss space.

Third, at least one of the plurality of grip portions has the both ends extend so as to surround the corresponding tooth and the tooth adjacent to the corresponding tooth.

Therefore, since at least one of the grip portions is formed such that at least one of the grip portions surrounds the plurality of teeth adjacent to each other, the spacer is capable of being more tightly attached to the teeth, and the teeth arrangement may be more stably maintained by more effectively supporting the teeth adjacent to the tooth loss space.

Fourth, the body portion is configured as a single structure, and is manufactured by laser cutting the base material formed of the shape memory alloy material on the basis of 3D scanning data of the teeth arrangement, or is manufactured by 3D printing the shape memory material on the basis of the 3D scanning data of the teeth arrangement.

Therefore, as the manufacturing process is significantly reduced and the configuration of the spacer is simplified, ease of manufacturing the spacer is significantly increased and the manufacturing time is significantly reduced. In addition, since the spacer is formed of the shape memory material and is configured as the single structure, the spacer of the present disclosure has more excellent durability than a spacer having an assembling structure, and the tooth loss space may be more effectively maintained due to the shape restoring characteristic of the spacer of the present disclosure.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a view illustrating a state in which a spacer according to an embodiment of the present disclosure is attached to teeth;

FIG. 2 is a perspective view illustrating the spacer according to an embodiment of the present disclosure;

FIG. 3 is a view illustrating a state in which a tooth loss space is reduced;

FIG. 4 is a view illustrating a state in which the tooth loss space is restored and maintained by the spacer according to an embodiment of the present disclosure;

FIG. 5 is a view illustrating a positional difference of bridges according to the present disclosure, the bridges being respectively positioned at different sides of the upper and lower jaws in order to avoid an occlusal force;

FIG. 6 is a view illustrating a structural difference of the spacers according to an embodiment of the present disclosure, the spacers being respectively positioned at the upper and lower jaws;

FIG. 7 is a view illustrating an implant treatment process;

FIG. 8 is a view illustrating a state in which the spacer according to an embodiment of the present disclosure is attached to teeth for the implant treatment;

FIG. 9 is a plan view illustrating the spacer according to a second embodiment of the present disclosure;

FIG. 10 is a plan view illustrating the spacer according to a third embodiment of the present disclosure;

FIG. 11 is a flowchart illustrating a method of manufacturing the spacer according to an embodiment of the present disclosure;

FIG. 12 is a flowchart specifically illustrating an additional processing process according to an embodiment of the present disclosure; and

FIG. 13 is a view schematically illustrating a manufacturing process of the spacer according to an embodiment of the present disclosure.

### DETAILED DESCRIPTION OF THE INVENTION

Hereinafter, an embodiment of the present disclosure will be described in detail with reference to the accompanying drawings.

However, the spirit of the present disclosure is not limited to the presented embodiment, and by addition, modification, and removal of another component, other embodiments included within other degenerative inventions or the scope of the spirit of the present disclosure may be easily proposed.

FIG. 1 is a view illustrating a state in which a spacer according to an embodiment of the present disclosure is attached to teeth.

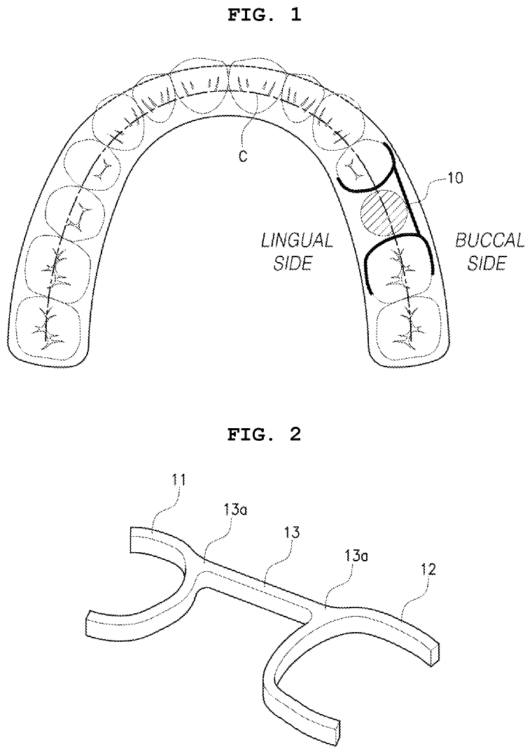

FIG. 2 is a perspective view illustrating the spacer according to an embodiment of the present disclosure.

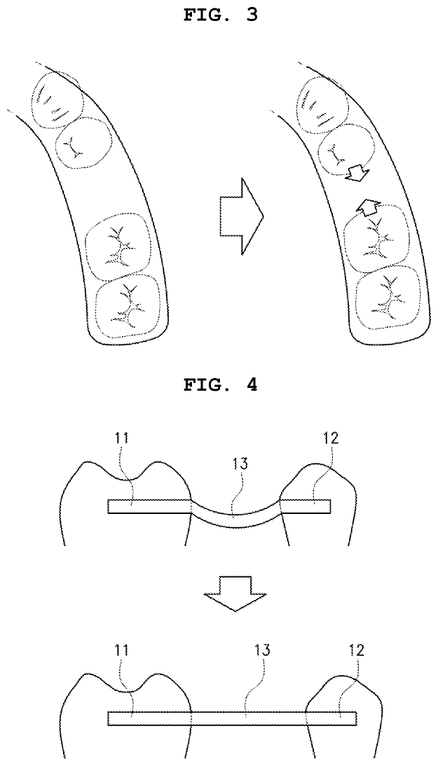

FIG. 3 is a view illustrating a state in which a tooth loss space is reduced.

FIG. 4 is a view illustrating a state in which the tooth loss space is restored and maintained by the spacer according to an embodiment of the present disclosure.

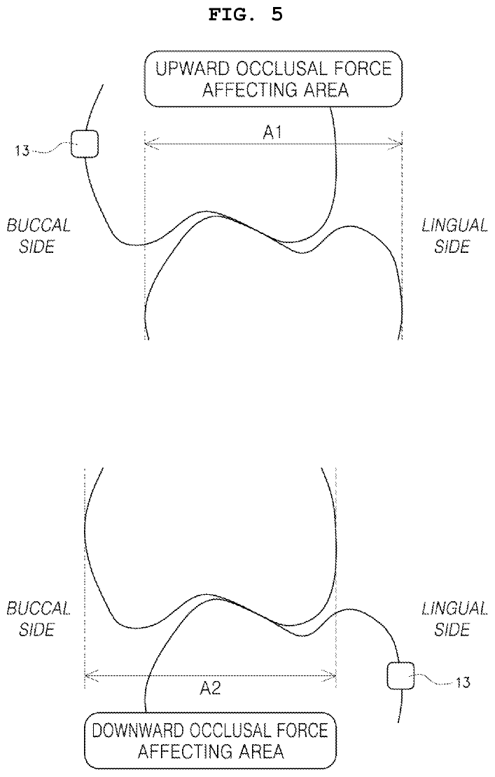

FIG. 5 is a view illustrating a positional difference of bridges according to the present disclosure, the bridges being respectively positioned at different sides of the upper and lower jaws in order to avoid an occlusal force.

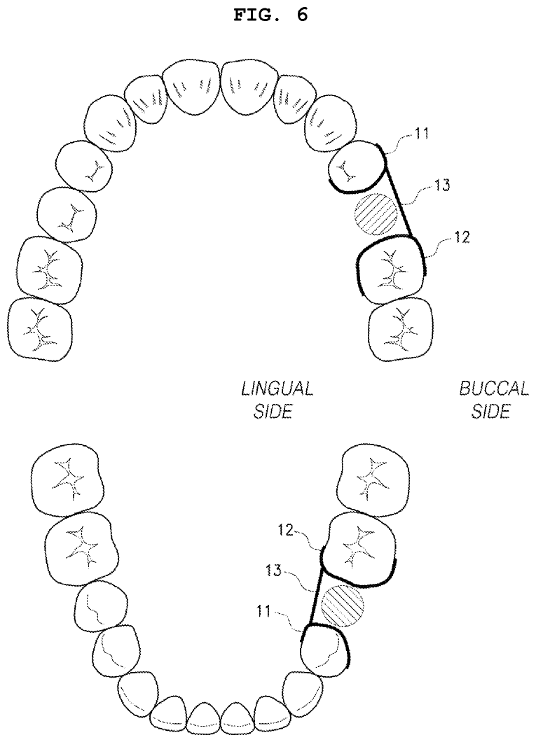

FIG. 6 is a view illustrating a structural difference of the spacers according to an embodiment of the present disclosure, the spacers being respectively positioned at the upper and lower jaws.

A spacer according to an embodiment of the present disclosure may be applied to various treatments that require maintenance of a tooth loss space from which a tooth is lost.

For example, when a tooth loss space is formed since a deciduous tooth is escaped before a permanent tooth is erupted, the spacer 1 may be applied to protect a space where the permanent tooth is erupted.

Alternatively, when a tooth is permanently lost and an artificial tooth implant treatment such as an implant treatment is required to be performed, the spacer 1 may be applied to prevent a reduction in size of a tooth loss space and to maintain the tooth loss space during a treatment period.

The spacer 1 according to an embodiment of the present disclosure may include a body portion 10 supporting teeth positioned at both sides of the tooth loss space in order to maintain the tooth loss space from which a tooth is lost in a patient's teeth arrangement.

The body portion 10 may be formed as a single integrated structure, and may be formed of a shape memory material.

For example, the body portion 10 may be formed of a shape memory metal material, and may specifically be formed of nitinol.

Alternatively, the body portion 10 may be formed of a shape memory polymer material or a composite material having shape memory properties.

According to characteristics of the material, the body portion 10 has properties to return to an original shape of the body portion 10, and may be formed such that the body portion 10 is capable of being elastically deformed.

The body portion 10 may include a first grip portion 11 surrounding a part around a tooth disposed at a first side with respect to the tooth loss space, a second grip portion 12 surrounding a part around a tooth disposed at a second side with respect to the tooth loss space, and a bridge 13 connecting the first grip portion 11 and the second grip portion 12 to each other.

The first grip portion 11 and the second grip portion 12 may be formed such that the first grip portion 11 and the second grip portion 12 respectively surround teeth corresponding to the tooth loss space.

In addition, in a state in which the first grip portion 11 and the second grip portion 12 surround the corresponding teeth, the first grip portion 11 and the second grip portion 12 may be adhered and fixed to the corresponding teeth by an adhesive.

Specifically, the first grip portion 11 and the second grip portion 12 may be formed such that the first grip portion 11 and the second grip portion 12 surround inner teeth surfaces toward the tooth loss space, lingual teeth surfaces, and buccal teeth surfaces of the corresponding teeth.

At this time, the first grip portion 11 and the second grip portion 12 may be formed such that the first grip portion 11 and the second grip portion 12 surround at least half of perimeters of the lingual teeth surfaces and the buccal teeth surfaces of the corresponding teeth in order to secure adhesion stability of the first grip portion 11 and the second grip portion 12. Preferably, the first grip portion 11 and the second grip portion 12 may be formed such that the first grip portion 11 and the second grip portion 12 surround ½ to ⅔ of the perimeters of the lingual teeth surfaces and the buccal teeth surfaces.

That is, the first grip portion 11 and the second grip portion 12 may be formed in curved shapes which surround the corresponding teeth from portions around the tooth loss space and which are open to opposite portions around the tooth loss space.

The bridge 13 may be formed in a bar shape that extends in a first direction, and a first end of the bridge 13 may be connected to the first grip portion 11 and a second end of the bridge 13 may be connected to the second grip portion 12.

The bridge 13 may be formed such that the bridge 13 is capable of being elastically deformed. Furthermore, the bridge 13 may be elastically deformed when the first grip portion 11 and the second grip portion 12 are close to each other, and a restoring force may be applied to the bridge 13 so that the first grip portion 11 and the second grip portion 12 move away from each other.

In order for the bridge 13 to not interfere with an erupting tooth or an implanted tooth, the bridge 13 may be formed such that the bridge 13 is lateralized to a lingual side or a buccal side from a center of the tooth loss space.

That is, the bridge 13 may be configured such that the bridge 13 is connected to a position lateralized to the lingual side or the buccal side from a center portion that corresponds to the center of the tooth loss space of the first grip portion 11 and the second grip portion 12.

More specifically, the bridge 13 may be lateralized to the lingual side of the buccal side with respect to a centerline C that extends along a center of the teeth arrangement illustrated in FIG. 1.

At this time, the tooth loss space may be defined as a space where a tooth that will be implanted or erupted will be positioned, and the bridge 13 may be configured such that the bridge 13 crosses an outer side of the tooth loss space.

Therefore, when a tooth is implanted or erupted in the tooth loss space, interference between the bridge 13 and the tooth may not occur.

Meanwhile, the body portion 10 may be formed such that the body portion 10 generally has a thickness and a height of 0.3 mm or more to 0.55 mm or less. That is, each cross-sectional area of the grip portions 11 and 12 and the bridge 13 may have a width and a height within a range of 0.3 mm to 0.55 mm.

Meanwhile, each thickness of end portions of the bridge 13 in contact with the grip portions 11 and 12 may be reinforced so that sharp engraved corners are not formed between the bridge 13 and the grip portions 11 and 12.

Specifically, ribs 13a having both sides rounded may be respectively formed on the end portions of the bridge 13 in contact with the grip portions 11 and 12. At this time, thickness reinforcement of the end portions of the bridge 13 may be performed within a range in which the thickness of the end portions of the bridge 13 does not exceed 0.55 mm.

Therefore, since the sharp engraved corners are not formed on contact portions where the grip portions 11 and 12 and the bridge 13 are in contact with each other, so that fracture may be prevented from occurring. In addition, the bridge 13 may more stably support the grip portions 11 and 12, and a pressure of the bridge 13 may be more effectively transferred to the grip portions 11 and 12.

Meanwhile, when the spacer 1 is applied to the upper jaw, the bridge 13 may be applied such that the bridge 13 is lateralized to the buccal side so as to avoid an occlusal force.

On the other hand, when the spacer 1 is applied to the lower jaw, the bridge 13 may be applied such that the bridge 13 is lateralized to the lingual side so as to avoid the occlusal force.

Specifically, referring to FIG. 5, teeth on the lower jaw are more lateralized to the lingual side than teeth on the upper jaw due to the structure of human teeth.

In addition, when the teeth on the upper jaw and the teeth on the lower jaw occlude with each other so as to bite food, an upward occlusal force directly affects an upward occlusal force affecting area A1 that corresponds to a vertical upward area of the teeth on the lower jaw. That is, the upward occlusal force directly affects an area lateralized to the lingual side from an occlusal center of the teeth on the upper jaw and the teeth on the lower jaw.

In addition, a downward occlusal force directly affects a downward occlusal force affecting area A2 that corresponds to a vertical downward area of the teeth on the upper jaw. That is, the downward occlusal force directly affects an area lateralized to the buccal side from the occlusal center.

Therefore, when the spacer 1 is applied to the upper jaw, the bridge 13 is applied such that the bridge 13 is lateralized to the buccal side, so that the influence of the occlusal force may be minimized. Furthermore, the breakage of spacer 1 may be prevented, and the spacer 1 may be prevented from being escaped from the teeth.

Therefore, when the spacer 1 is applied to the lower jaw, the bridge 13 is applied such that the bridge 13 is lateralized to the lingual side, so that the influence of the occlusal force may be minimized. Furthermore, the breakage of spacer 1 may be prevented, and the spacer 1 may be prevented from being escaped from the teeth.

However, in consideration of the ease of treatment, the spacer 1 may be applied such that the bridge 13 is lateralized to the lingual side regardless of the upper jaw or the lower jaw.

For example, when the spacer 1 is applied to the implant treatment, the spacer 1 may be applied such that the bridge 13 is lateralized to the lingual side regardless of the upper jaw or the lower jaw.

Hereinafter, referring to the implant treatment process, an example in which the spacer 1 is applied such that the bridge 13 is lateralized to the lingual side will be described.

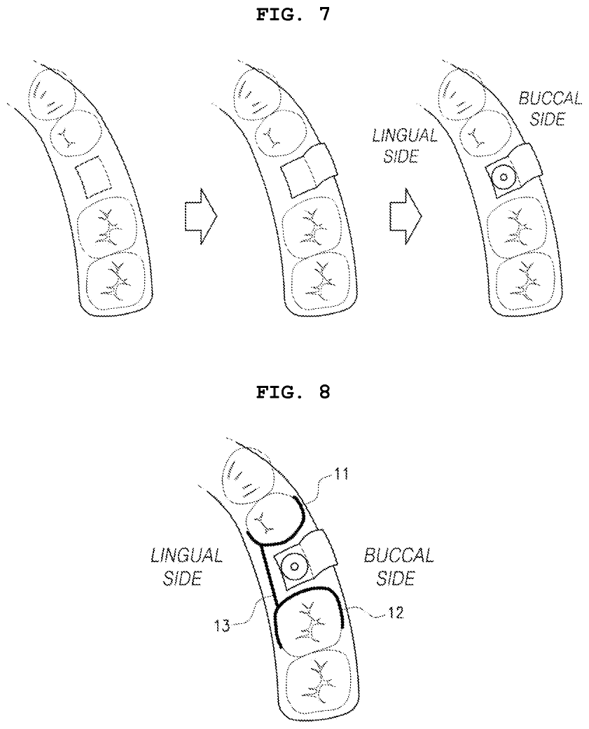

FIG. 7 is a view illustrating an implant treatment process.

FIG. 8 is a view illustrating a state in which the spacer according to an embodiment of the present disclosure is attached to teeth for the implant treatment.

Referring to FIG. 7, in order to perform an implant treatment, a gingiva flap at a position where the implant treatment is performed is incised and is turned over toward the buccal side, and then an implant structure is mounted at a position where the flap is removed.

At this time, as the flap is turned over toward the buccal side, when the bridge 13 of the spacer 1 crosses the buccal side of the tooth loss space, the bridge 13 may interfere with a surgical instrument that enters from the buccal side, so that difficulties may occur while the treatment is performed.

Therefore, when an artificial structure is required to be mounted in the position of the tooth loss space, such as the implant treatment, or a treatment using various medical apparatuses is required to be performed, the bridge 13 may be applied such that the bridge is lateralized to the lingual side regardless of the upper jaw or the lower jaw.

Meanwhile, in the description of the present disclosure as described above, an example in which the grip portions are formed such that the grip portions surround a part of a single tooth and the single bridge is applied is described, but the spacer of the present disclosure is not limited to an embodiment described above.

Specifically, in order for a more stable support of teeth and for reinforcing adhesion stability, at least one of the first grip portion 11 and the second grip portion 12 may surround a plurality of teeth adjacent to each other.

In addition, in order for the more stable support of teeth and for reinforcing durability, a plurality of bridges 13 may be applied such that the plurality of bridges 13 is spaced apart from each other.

Hereinafter, another embodiment of the spacer according to the present disclosure will be described in detail with reference to the drawings.

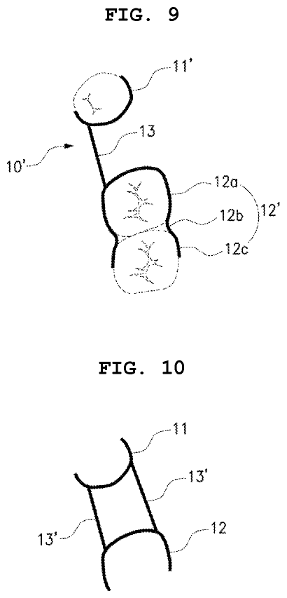

FIG. 9 is a plan view illustrating the spacer according to a second embodiment of the present disclosure.

The spacer 1′ according to a second embodiment of the present disclosure only has a difference in a shape of the grip portions of the spacer 1 according to a first embodiment described above, so that the same configurations will be briefly described by using the names and the reference numerals of the same configurations and the difference in the shape of the grip portions will be described in detail.

Meanwhile, the spacer 1′ according to the second embodiment of the present disclosure may include a body portion 10′ supporting teeth positioned at both sides of the tooth loss space in order to maintain the tooth loss space from which the tooth is lost in the patient's teeth arrangement.

The body portion 10′ may include a first grip portion 11′ surrounding a part around a tooth disposed at a first side with respect to the tooth loss space, a second grip portion 12′ surrounding a part around a tooth disposed at a second side with respect to the tooth loss space, and the bridge 13 connecting the first grip portion 11′ and the second grip portion 12′ to each other.

At this time, at least one of the first grip portion 11′ and the second grip portion 12′ may be formed such that at least one of the first grip portion 11′ and the second grip portion 12′ surround the corresponding tooth and another tooth positioned adjacent to the corresponding tooth along the teeth arrangement.

That is, at least one of the first grip portion 11′ and the second grip portion 12′ may be formed such that at least one of the first grip portion 11′ and the second grip portion 12′ surround a plurality of tooth at the corresponding position.

Hereinafter, for convenience of explanation, a structure in which the first grip portion 11′ is configured as same as the first grip portion 11 of the first embodiment that is described above and only the second grip portion 12′ is formed such that the second grip portion 12′ surrounds the plurality of teeth will be described as an example.

Referring to FIG. 9, the second grip portion 12′ extends in a curved shape such that the second grip portion 12′ surrounds the inner teeth surfaces toward the tooth loss space, the lingual teeth surfaces, and the buccal teeth surfaces of the corresponding tooth. Furthermore, the second grip portion 12′ may further extend such that both ends of the second grip portion 12′ surround lingual teeth surfaces and the buccal teeth surfaces of another tooth adjacent to the corresponding tooth.

In addition, a portion in the second grip portion 12′ corresponding to an interdental portion may protrude toward the interdental portion so that the portion in the second grip portion 12′ is inserted into the interdental portion. Therefore, a space between the second grip portion 12′ and the interdental portion is minimized, so that a problem that foreign substances are stuck between the second grip portion 12′ and the interdental portion may be effectively prevented.

That is, the second grip portion 12′ may include a first portion 12a surrounding the inner teeth surfaces toward the tooth loss space of the corresponding tooth, the lingual teeth surfaces, and the buccal teeth surfaces, a second portion 12b which extends along the teeth arrangement from both ends of the first portion 12a and which is positioned so as to correspond to the interdental portion between the corresponding tooth and an adjacent tooth adjacent to the corresponding tooth, the second portion 12b which protrudes and which is curved such that the second portion 12b is inserted into the interdental portion, and a third portion 12c which extends along the teeth arrangement from both ends of the second portion 12b and which surrounds the lingual teeth surfaces and the buccal teeth surfaces of the adjacent tooth.

At this time, the third portion 12c may extend such that the third portion 12c surrounds ½ to ⅔ of a perimeter of the lingual teeth surface and the buccal teeth surface.

According to the spacer 1′ of the second embodiment of the present disclosure, since at least one of the plurality of grip portions surrounds the plurality of teeth adjacent to each other, the spacer 1′ is capable of being more tightly attached to the teeth. Furthermore, since the spacer 1′ more effectively supports the teeth adjacent to the tooth loss space, the tooth loss space is more effectively prevented from being reduced, and the teeth arrangement may be stably maintained.

In addition, since a portion of the spacer 1′ passing the interdental portion of the plurality of teeth protrudes such that the portion of the spacer 1′ is inserted into the interdental portion, a gap between the grip portions and the interdental portion is minimized, so that a problem such as foreign substances sticking may be effectively prevented.

FIG. 10 is a plan view illustrating the spacer according to a third embodiment of the present disclosure.

A spacer 1″ according to a third embodiment of the present disclosure is different from the spacer 1 according to the first embodiment described above in the number of bridges, so that the same configurations will be briefly described by using the names and the reference numerals of the same configurations and the difference in the bridge will be described in detail.

the spacer 1″ according to the third embodiment of the present disclosure may include a body portion 10″ supporting teeth positioned at both sides of the tooth loss space in order to maintain the tooth loss space from which the tooth is lost in the patient's teeth arrangement.

The body portion 10″ may include the first grip portion 11 surrounding a part around a tooth disposed at a first side with respect to the tooth loss space, the second grip portion 12 surrounding a part around a tooth disposed at a second side with respect to the tooth loss space, and a plurality of bridges 13′ connecting the first grip portion 11 and the second grip portion 12 to each other.

The plurality of bridges 13′ may be formed in a bar shape that extends in a first direction, and a first end of each of the plurality of bridges 13′ may be connected to the first grip portion 11 and a second end of each of the plurality of bridges 13′ may be connected to the second grip portion 12.

In order for the plurality of bridges 13′ to not interfere with an erupting tooth or an implanted tooth, the plurality of bridges 13′ may be formed such that the plurality of bridges 13′ is lateralized to a lingual side or a buccal side from a center of the tooth loss space.

Specifically, two bridges 13′ may be applied, and one bridge 13′ may be configured such that the one bridge 13′ is connected to a position lateralized to the lingual side from the center portion corresponding to the center of the tooth loss space of the first grip portion 11 and the second grip portion 12 so that the one bridge 13′ crosses the tooth loss space from the lingual side.

That is, another bridge 13′ may be configured such that the another bridge 13′ is connected to a position lateralized to the buccal side from the center portion corresponding to the center of the tooth loss space of the first grip portion 11 and the second grip portion 12 so that the another bridge 13′ crosses the tooth loss space from the buccal side.

Therefore, as the two bridges 13′ are spaced apart from each other and the tooth loss space is completely open through a space between the two bridges 13′, interference between the erupting tooth or the implanted tooth with the bridges 13′ may be prevented from occurring.

According to the spacer 1″ of the third embodiment of the present disclosure, as the plurality of bridges 13′ generating a force maintaining the tooth loss space by supporting the plurality of grip portions is provided, the tooth may be more stably supported and durability may be reinforced, and treatment stability may be improved since a function of the spacer 1″ is not lost even if one part of the bridge is broken.

Meanwhile, the body portion of the spacer of an embodiment of the present disclosure may be manufactured of a shape memory material on the basis of 3D scanning data of the teeth arrangement, and various methods capable of manufacturing the spacer of the present disclosure as a single configuration may be applied.

For example, the spacer may be manufactured by performing 3D printing with the shape memory material on the basis of the 3D scanning data of the teeth arrangement.

Specifically, by using design tools such as CAD and CAM, a 3D model of the spacer may be designed in consideration of the tooth loss space at a position where the spacer is required to be mounted and a shape and an arrangement of adjacent teeth in the 3D scanning data of the teeth arrangement.

In addition, by using a 3D printer, the spacer corresponding to the 3D model may be manufactured by printing the spacer with the shape memory material.

Various shape memory materials capable of being used in a known 3D printer may be used as the shape memory material used in the 3D printer, and a technology of printing an object corresponding to a 3D model by using the 3D printer and the shape memory material is variously disclosed in a known technology, so that the detailed description thereof will be omitted.

Alternatively, the spacer may be manufactured by being laser cut from a base material of a shape memory alloy material on the basis of the 3D scanning data of the teeth arrangement.

In addition, after the shape memory alloy material is laser cut, the body portion 10 may be manufactured by applying an additional processing for shape deformation.

In addition, after the shape memory alloy material is laser cut, the body portion 10 may be manufactured by performing a thermal treatment. Therefore, the body portion 10 may have a higher hardness, and a shape of the body portion 10 may be stabilized.

In addition, the body portion 10 may be manufactured by performing polishing on a surface of the body portion 10. Therefore, the body portion 10 may have a smoother surface, and may generally be formed in a safe structure since a sharp portion is removed.

Hereinafter, a method of manufacturing the spacer according to an embodiment of the present disclosure by laser cutting the spacer from the base material of the shape memory alloy material will be described in detail with reference to the drawings.

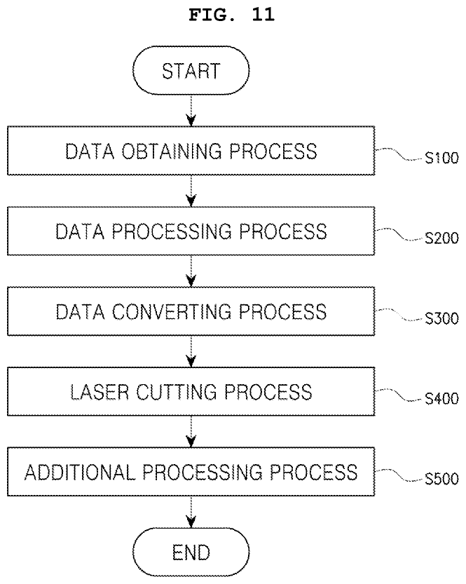

FIG. 11 is a flowchart illustrating a method of manufacturing the spacer according to an embodiment of the present disclosure.

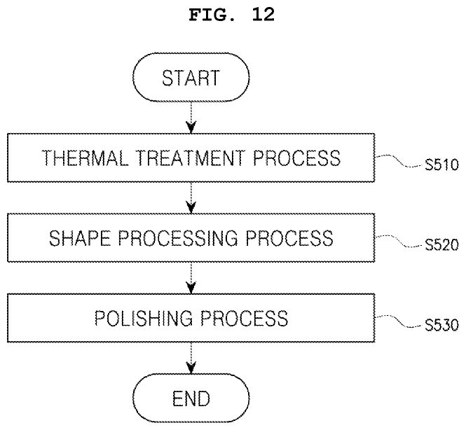

FIG. 12 is a flowchart specifically illustrating an additional processing process according to an embodiment of the present disclosure.

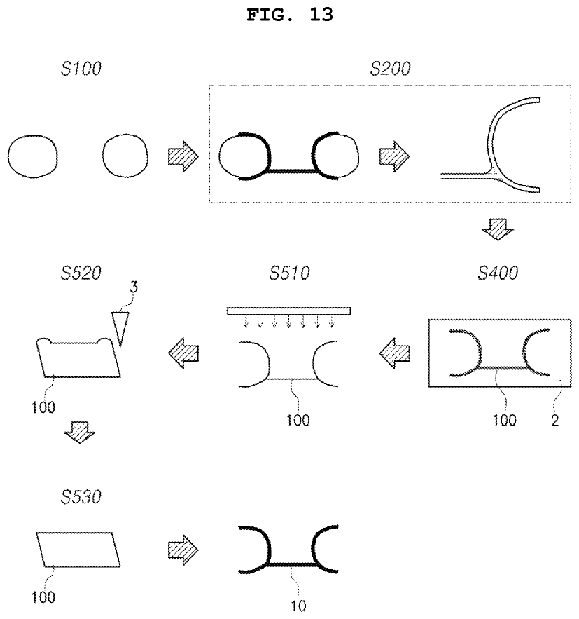

FIG. 13 is a view schematically illustrating a manufacturing process of the spacer according to an embodiment of the present disclosure.

A method of manufacturing a spacer according to an embodiment of the present disclosure may be applied to all spacers of the first embodiment, the second embodiment, and the third embodiment that are described above.

The method of manufacturing the spacer according to an embodiment of the present disclosure may include a data obtaining process obtaining scanning data of a teeth arrangement including a tooth loss space, a data processing process processing the scanning data, a data converting process converting modified data to data for laser processing; a cutting process laser cutting a base material, and an additional processing process additionally processing a body portion forming body that is laser cut.

First, by using a 3D scanning device, 3D scanning data of a teeth arrangement including an affected area where the spacer is required to be applied may be obtained.

At this time, scanning data refer to photographed data of the teeth arrangement of the upper jaw or the lower jaw where the body portion 10 is attached, the photographed data being photographed by using a 3D camera.

The scanning date may include tooth loss space information and teeth arrangement information.

In addition, the scanning data may further include an interdental shape formed in the teeth arrangement, and a curvature, a gradient, and so on of a tooth surface of each of teeth.

The scanning data may be a file corresponding to an stl formatted file. [S100, Data Obtaining Process]

After the data obtaining process, the data processing process for generating a processing line defining a laser cutting shape so as to manufacture the body portion 10 on the basis of the scanning data may be performed.

After the scanning data is obtained, in the scanning data, a pair of grip portion processing lines for defining cutting shapes of the first grip portion 11 and the second grip portion 12 may be generated in teeth respectively positioned at the both sides with respect to the tooth loss space of the tooth.

The grip portion processing lines may be generated along the inner teeth surfaces toward the tooth loss space, the lingual teeth surfaces, and the buccal teeth surfaces of the corresponding teeth, and may be generated while including a pair of processing lines which is spaced apart from each other and which is parallel to each other so as to correspond to a thickness required for setting a thickness.

In addition, a bridge processing line for defining a cutting shape of the bridge 13 may be generated by connecting the pair of grip portion processing lines to each other.

The bridge processing line may be generated such that the bridge processing line is connected to a portion lateralized to the lingual side or the buccal side from a center of the pair of grip portion processing lines, and may be generated such that the bridge 13 is manufactured in a shape lateralized to the lingual side or the buccal side so as to avoid the tooth loss space.

The bridge processing line may include a pair of processing lines which is spaced apart from each other and which is parallel to each other so as to correspond to a thickness required for setting a thickness.

In addition, end portions of the bridge processing line in contact with the grip portion processing lines may be rounded in both sides thereof so that the ribs 13a are formed.

As the grip portion processing lines are generated along the teeth surfaces of the corresponding teeth during the data processing process, the teeth surface shape of the teeth may be reflected and generated. [S200, Data Processing Process]

When the data processing process is finished, the generated processing line data may be converted into the data for laser processing.

For example, the processing line data may be saved as two-dimensional data.

In addition, by using a program automatically generating a movement path of a laser cutting device on the basis of the two-dimensional data, the processing line data may be converted into the data for laser processing.

The generated data for laser processing may be a two-dimensional CAD file reflecting the processing line data. [S300, Data Converting Process]

When the data converting process is finished, the data for laser processing may be input into the laser cutting device for laser processing.

In addition, the laser cutting device is operated according to the data for laser processing, and an input base material 2 may be laser cut. That is, the laser cutting device may laser cut the base material 2 on the basis of the data for laser processing.

Specifically, the base material 2 may be provided in a flat plate shape formed of the shape memory alloy material. That is, the base material 2 may be a plate formed of the shape memory alloy material. For example, the base material 2 may be a nitinol thin plate.

The laser cutting device may be provided with a jig in which the base material 2 is accommodated and fixed. In addition, the base material 2 may be accommodated and fixed in the jig.

A setting value may be input to the laser cutting device before performing laser cutting. For example, an operator may set a setting value such as a processing speed of 200 mm/s, a processing acceleration of 3000 mm/s, a pulse power in a pulse type of 300 w, a pulse frequency of 600 Hz, a pulse time of 400 us, an argon gas pressure of 3.0 mpa, and so on.

After the base material 2 is input and the setting value of the laser cutting device is set, the operator may operate the laser cutting device.

When the laser cutting device is operated, cutting of the base material 2 is performed along the processing lines, and a body portion forming body 100 having a basic shape of the body portion 10 may be cut and formed. [S400, Laser Cutting Process]

After the laser cutting process is performed, the additional processing process additionally processing the base material 2 in which the body portion forming body 100 is formed may be performed.

During the additional processing process, a mechanical processing or a chemical processing performed so that the body portion forming body 100 has a final shape of the body portion 10 may be performed.

For example, the additional processing process may include a thermal treatment process for stabilizing the shape of the body portion forming body 100.

In addition, the additional processing process may include a shape processing process reflecting teeth surface shapes to contact surface forming surfaces of the body portion forming body 100 that forms contact surfaces of the grip portions in contact with the teeth surfaces.

In addition, the additional processing process may include a polishing process polishing the body portion forming body 100 in which the shape processing process is completed.

The working order of the thermal treatment process, the shape processing process, and the polishing process is not limited to the listed order, and is capable of being variously changed according to a work efficiency. For example, the polishing process may be performed after the shape processing process, and the thermal treatment process may be performed after the polishing process. Alternatively, the thermal treatment process is performed after the shape processing process, and then the polishing process may be performed.

Hereinafter, an example in which the shape processing process is performed after the thermal treatment process and the polishing process is performed after the shape processing process will be described in detail. [S500, Additional Processing Process]

After the body portion forming body 100 is cut from the base material 2 and then fixed to a cutting space of the base material 2 or fixed to a separate jig, the body portion forming body 100 may be thermally treated by being input into a thermal treatment device.

After the body portion forming body 100 is thermally treated, the body portion forming body 100 may be cooled by being input into a cooling device or may be naturally cooled. [S510, Thermal Treatment Process]

After the body portion forming body 100 is cut from the base material 2, the body portion forming body 100 may be input so as to perform the shape processing process.

The shape processing process may be performed by various tools, devices, and so on that are capable of cutting the surface of the body portion forming body 100. For example, the shape processing process may be performed by a bur 3 having a cone shape.

Due to characteristics of laser processing, the gradient or the curvature in a vertical direction may be difficult to be formed on the cutting surfaces. Therefore, by using a cutting tool or a cutting device, the gradient or the curvature considering the teeth surface shape may be processed on the cutting shape forming surfaces of the body portion forming body 100.

For example, the contact surface forming surfaces of the body portion forming body 100 may be processed by using the bur 3.

At this time, the contact surface forming surfaces of the body portion forming body 100 may be processed corresponding to the shape of the teeth surfaces of the teeth on the basis of the scanning data.

For example, when the contact surface forming surfaces of the body portion forming body 100 correspond to the teeth surfaces inclined upward to the tooth loss space, the contact surface forming surfaces of the body portion forming body 100 may be processed in a shape inclined downward to the corresponding teeth surfaces.

In addition, when the contact surface forming surfaces of the body portion forming body 100 correspond to the teeth surfaces inclined downward to the tooth loss space, the contact surface forming surfaces of the body portion forming body 100 may be processed in a shape inclined upward to the corresponding teeth surfaces.

In addition, when the contact surface forming surfaces of the body portion forming body 100 correspond to the curved teeth surfaces, the contact surface forming surfaces of the body portion forming body 100 may be processed in a shape curved corresponding to the curved teeth surfaces. [S520, Shape Processing Process]

After the shape processing process is performed, the polishing process polishing the body portion forming body 100 may be performed.

In the polishing process, polishing processing of the body portion forming body 100 may be performed by using various polishing tools and devices that are capable of polishing the surface of the body portion forming body 100.

A method of polishing a plate of a shape memory alloy material is variously disclosed in a known technology, the detailed description thereof will be omitted.

The body portion 10 may have a smoother surface by performing the polishing process, and may generally be formed in a safe structure since a sharp portion is removed.

Specifically, a flash caused by a molten base material may be formed on the body portion forming body 100 during the laser cutting process. In addition, a burring may be formed on a processing portion during the shape processing process.

The flash, the burring, and the corner portions are processed by the polishing process, so that the body portion forming body 100 may have the smoother surface, and the sharp portion may be removed. [S530, Polishing Process]

According to the spacer of an embodiment of the present disclosure, the body portion of the spacer is manufactured by being 3D printed or by laser cutting the plate of the shape memory alloy material on the basis of the scanning data of the teeth arrangement, and the spacer is famed in a single structure, so that there are advantages that the spacer is easily manufactured and the manufacturing time is significantly reduced.

In addition, the plurality of grip portions supporting teeth at the both sides with respect to the tooth loss space is formed such that the plurality of grip portions surrounds parts around the corresponding teeth. Specifically, the plurality of grip portions is formed such that the plurality of grip portions is open to the opposite sides of the tooth loss space. Therefore, unlike a conventional spacer that surrounds the entire tooth circumference, the spacer of the present disclosure does not require an additional treatment process for securing a space where the spacer is mounted between adjacent teeth. Accordingly, problems such as occurrence of discomfort of a patient due to an additional treatment process and a prolonged treatment period may be effectively solved.

In addition, as the bridge connecting the plurality of grip portions is formed such that the bridge crosses the outer side of the tooth loss space by avoiding the tooth loss space, there is an effect that interference between an erupting tooth or an implanted tooth in the tooth loss space and the bridge is not occurring.

In addition, according to the spacer of the first embodiment of the present disclosure, the bridge at the upper jaw is lateralized to the buccal side and the bridge at the lower jaw is lateralized to the lingual side so as to avoid the occlusal force. Therefore, as the effect of the occlusal force is minimized, the breakage of the spacer by the occlusal force may be effectively prevented and the spacer may be effectively prevented from being escaped from the teeth.

In addition, according to the spacer of the second embodiment of the present disclosure, since at least one of the grip portions is formed such that at least one of the grip portions surrounds the plurality of teeth adjacent to each other, the spacer is capable of being more tightly attached to the teeth, and the teeth arrangement may be more stably maintained by more effectively supporting the teeth adjacent to the tooth loss space.

In addition, according to the spacer of the third embodiment of the present disclosure, as the plurality of bridges is provided, there are effects that the teeth may be more stably supported, durability of the spacer may be reinforced, and treatment stability may be improved since the bridge does not lose the function even if a part of the bridge is broken.

However, according to the method of manufacturing the spacer of an embodiment of the present disclosure described above, since the body portion 10 is capable of being manufactured as a single integrated structure, the manufacturing cost of the spacer may be significantly reduced.

In addition, as the body portion 10 is manufactured by being laser cut on the basis of the 3D scanning data of the teeth arrangement, the time required for manufacturing the spacer may be significantly reduced.

In addition, as the time required for manufacturing the spacer is reduced, the reduction in the tooth loss space during a spacer manufacturing time is minimized. Therefore, an attachment time may be reduced since the manufactured spacer and the teeth are stably matched, and the treatment period may be reduced.

In addition, due to characteristics of laser processing, the gradient or the curvature in the vertical direction may be difficult to be formed on the cutting surfaces. However, in the method of manufacturing the spacer according to an embodiment of the present disclosure, a processing line defining a laser cutting shape on the basis of the 3D scanning data of the teeth arrangement is generated, and then the body portion forming body 100 is formed by performing laser cutting along the processing line. Then, by performing a separate additional processing, the processing considering the teeth surface shape may be performed on the contact surface forming surfaces of the body portion forming body 100 that forms the teeth contact surface of the grip portions. Therefore, as the gradient and the curvature of the teeth surfaces are reflected on the teeth contact surfaces of the grip portions, the grip portions may be more closely attached to the teeth, and foreign substances sticking may be prevented and the adhesion stability may be significantly improved.

What is claimed is:

1. A spacer comprising:
a body portion configured as a single structure and formed of a shape memory material, the body portion supporting teeth at both sides of a tooth loss space,
wherein the body portion comprises:
a first grip portion surrounding a part around a tooth disposed at a first side with respect to the tooth loss space;
a second grip portion surrounding a part around a tooth disposed at a second side with respect to the tooth loss space; and
a bridge connecting the first grip portion and the second grip portion to each other, and
the first grip portion and the second grip portion have curved shapes open toward directions opposite to each other.

2. The spacer of claim 1, wherein the bridge is disposed to be lateralized from center portions of the first grip portion and the second grip portion such that the bridge crosses an outer side of the tooth loss space.

3. The spacer of claim 1, wherein at least one of the first grip portion and the second grip portion has both ends that extend so as to surround a corresponding tooth and a tooth adjacent to the corresponding tooth together.

4. The spacer of claim 1, wherein the body portion is manufactured by laser cutting a base material formed of a shape memory alloy material on the basis of 3D scanning data of a teeth arrangement, or is manufactured by 3D printing the shape memory material on the basis of the 3D scanning data of the teeth arrangement.

5. A method of manufacturing a spacer, the spacer comprising a plurality of grip portions formed such that the plurality of grip portions surrounds parts around teeth disposed at both sides of a tooth loss space so as to maintain the tooth loss space, the spacer comprising a bridge connecting the plurality of grip portions to each other, and the method comprising:
a data obtaining process obtaining 3D scanning data of a teeth arrangement comprising the tooth loss space by using a 3D scanning device;
a data processing process generating a plurality of grip portions processing lines surrounding the parts around the teeth disposed at the both sides of the tooth loss space and generating a bridge processing line connecting the plurality of grip portions processing lines to each other, on the 3D scanning data; and
a laser cutting process laser cutting a base material having a flat plate shape formed of a shape memory alloy material on the basis of processing line data comprising the plurality of grip portions processing lines and the bridge processing line, thereby forming a body portion forming body having a basic shape of the body portion on the base material.
